(19) United States
(12) Patent Application Publication
(10) Pub. No.: US 2007/0184076 A1
(43) Pub. Date: Aug. 9, 2007

(54) Liquid-filled nanodroplets for anti-cancer therapy

(76) Inventors: Evan C. Unger, Tucson, AZ (US); Terry O. Matsunaga, Tucson, AZ (US); Reena Zutshi, Tucson, AZ (US)

Correspondence Address: DLA PIPER US LLP, 4365 EXECUTIVE DRIVE, SUITE 1100, SAN DIEGO, CA, 92121-2133, US

(21) Appl. No.: 11/349,660

(22) Filed: Feb. 7, 2006

(51) Int. Cl. A61K 9/127; A61K 9/00

(52) U.S. Cl. 424/400; 424/450; 977/907

(57) ABSTRACT

A nanodroplet composition is provided, the nanodroplets include a lipid encapsulating a biologically compatible oil, a fluorocarbon composition including one or more fluorinated hydrocarbons, and a therapeutically active compound, where the fluorocarbon composition is in a liquid state at a temperature that is equal to, or lower than, the body temperature of a mammal.

### BACKGROUND

1. Field of the Invention

This invention relates to nanodroplets, more particularly to nanodroplets filled with biocompatible oil and fluorinated hydrocarbons that are liquid at or below the body temperature of a mammal. The nanodroplets may be used in therapeutic delivery systems.

2. Background

Targeted drug delivery is important in many applications, for example where toxicity of a drug, if delivered systemically, is an issue. Targeted drug delivery may help eliminate or at least minimize toxic side effects and lower the required dosage amounts, among other beneficial features.

The known methods and materials that are used for introduction of many therapeutic agents, such as genetic materials, living cells, or some synthetic drugs, are sometimes of limited value. For example, various mechanisms that have been tried to deliver genetic material to living cells (e.g., calcium phosphate precipitation and electroporation, and using carriers such as cationic polymers and aqueous-filled liposomes) have at times revealed relative inefficiency in vivo and limited value for cell culture transfection. These methods may not easily allow local release, delivery and integration of genetic material to the target cell.

One important limitation of the previously tried delivery methods, as applicable to genetic materials, has been the fact that difficulties may arise with delivering the genetic material from the extracellular space to the intracellular space. Even the effective localization of genetic material at the surface of selected cell membranes has turned out to be difficult.

A variety of techniques have been tried in vivo (e.g., various viruses have been used as vectors to transfer genetic material to cells) but no sufficient success has been achieved. Despite extensive work on viral vectors, it has been difficult to develop a successfully targeted viral mediated vector for delivery of genetic material in vivo.

Other methods that have been tried include using a whole virus but without great success because of the inherent limitations on the amount of genetic material that may be placed inside of the viral capsule and also because of possibility of dangerous interactions that might be caused by live virus. While the essential components of the viral capsule may be isolated and used to carry genetic material to selected cells, other difficulties that are very difficult to overcome arise in vivo. For instance, in vivo, not only must the delivery vehicle recognize certain cells but it also must be delivered to these cells.

Conventional, liquid-containing liposomes have been tried for delivery of genetic material to cells in cell culture, but the degree of efficiency has been sometimes disappointing in vivo for cellular delivery of genetic material. For example, cationic liposome transfection techniques have not worked effectively in vivo. More effective means are needed to improve the cellular delivery of therapeutics such as genetic material. Gas- or gas precursor-filled liposomes have been tried as delivery vehicles, and while such vehicles provide promising results in some areas, the delivery of many synthetic drugs, in particular, hydrophobic drugs, may be inefficient due to the often limited solubility of the drugs.

Accordingly, better means of delivery for therapeutics such as synthetic drugs and genetic materials are desired to treat a wide variety of human and animal diseases; The present invention is directed to addressing the foregoing, as well as other important needs for the effective targeted delivery of therapeutics.

### SUMMARY

According to some embodiments of the invention, a composition is provided, including nanodroplets dispersed in an aqueous medium, where the nanodroplets include a lipid defining an inner area of the nanodroplets. This inner area includes a biologically compatible oil, a fluorocarbon composition including at least one fluorinated hydrocarbon, and a therapeutically active compound, where the fluorocarbon composition is in a liquid state at a temperature that is equal to, or lower than, the body temperature of a mammal.

According to some embodiments of the invention, a therapeutically active compound may be included in the nanodroplet composition. The therapeutically active compound, such as any synthetic drug or genetic material, may comprise at least one anti-cancer agent. Targeting ligands may be further incorporated into the nanodroplets, if desired.

### BRIEF DESCRIPTION OF THE FIGURES

FIG. 1 is a schematic illustration of a liquid-filled liposome according to one embodiment of the invention.

FIG. 2 shows schematically regularly and irregularly shaped nanodroplets.

### DETAILED DESCRIPTION

### A. Definitions

The following definitions apply.

The term “nanodroplets” refers to a group of small, generally spherically shaped particles of a liquid suspended in a medium, where the mean value of the diameter or quasi-diameter (as defined below) of the particles within the group is below about 1 μm.

The term “a quasi-diameter” applies to non-sphere shaped, i.e., not perfectly spherical nanodroplets and refers to the length of the longest of a plurality of straight lines connecting the following three points: the geometrical center of a nanodroplet, and two points on its surface.

The term “a lipid” refers to compounds of biological origin that are typically water-insoluble or nonpolar, including aliphatic, cyclic and aromatic compounds generally classified as fatty acids, fatty-acid derived phospholipids, sphingolipids, glycolipide waxes, and terpenoids, such as retinoids and steroids.

The term “lipophilic” refers to compounds having an affinity for lipids, or having tendency to react or to combine with lipids.

The term “a biologically compatible oil” refers to any oil capable of forming one or more biochemically stable systems when mixed with other compounds.

The term “a fluorinated hydrocarbon” refers to compounds derived from hydrocarbons, where one or more hydrogen atom(s) is (are) substituted with fluorine atom(s); accordingly, fluorinated hydrocarbons contain atoms of carbon and fluorine, and, optionally, also atoms of hydrogen. The term “a fluorinated hydrocarbon” includes fluorinated hydrocarbons derived from both saturated, unsaturated and cyclic hydrocarbons, including straight-chained and branched hydrocarbons.

The term “a CX fluorocarbon” refers to a fluorocarbon having a hydrocarbon chain comprising X atoms of carbon, the X atoms of carbon may be arranged in a straight-chain, branched or cyclic fashion and the hydrocarbon chain may be either saturated or unsaturated. To explain the term by way of illustration, “C6 fluorocarbon” (i.e., X=6) refers to a fluorocarbon having 6 carbons in the hydrocarbon chain; therefore, “C6 fluorocarbon” refers to fluorocarbons that include fully or partially fluorinated hexane, including n-hexane, and any branched hexane.

The term “a CX or higher fluorocarbon” refers to a fluorocarbon having a hydrocarbon chain comprising X or more atoms of carbon, the X atoms of carbon may be arranged in a straight-chain, branched or cyclic fashion and the hydrocarbon chain may be either saturated or unsaturated. To explain the term by way of illustration, “C6 fluorocarbon or higher” (i.e., X≧6) refers to a fluorocarbon having 6 more carbons in the hydrocarbon chain. Examples of “C6 fluorocarbon or higher” include “C6 fluorocarbon,” “C7 fluorocarbon” referring to fully or partially fluorinated heptane, including n-heptane, or any branched heptane, “C8 fluorocarbon” referring to fully or partially fluorinated octane, including n-octane, or any branched octane, and so forth.

The term “a fluorocarbon composition” refers to a composition that may include either a single fluorocarbon or a mixture of a plurality of fluorinated hydrocarbons.

The term “a perfluorinated hydrocarbon” refers to a kind of a fluorinated hydrocarbon in which every hydrogen atom is substituted with fluorine atoms; accordingly, “a perfluorinated hydrocarbon” refers to compounds comprising atoms of carbon and fluorine only. Perfluorinated hydrocarbons that may be used may be derived from both saturated, unsaturated and cyclic hydrocarbons, including straight-chained and branched hydrocarbons. One example of a class of perfluorinated hydrocarbons that may be used includes saturated perfluorinated hydrocarbons that may be described by a general formula CₙF₂ₙ₊₂, where n is an integer and n≧1.

The term “a therapeutically active compound” refers to a compound which, when administered to a mammal in need thereof, may elicit a beneficial therapeutic response. The term “an anti-cancer agent” refers to compounds and substances that may be used for the treatment of various cancers and other tumors.

The term “a targeting ligand” refers to a ligand bound to the lipid forming the nanodroplet. This targeting ligand assists the targeted delivery system in finding the targeted cells. A ligand may be any compound of interest which will bind to another compound, such as a receptor.

The term “a targeted cell” refers to a cell to which therapeutic agents, such as genetic materials, living cells, or synthetic drugs, are intended to be delivered.

The term “the targeted delivery system” refers to a composition that is intended for delivery to a particular type of cells, where the composition includes therapeutic agents, such as genetic materials, living cells, or synthetic drugs.

The term “solubilization” refers to a process of making a substance, which has no or low solubility in a solvent, soluble or more soluble than prior to the solubilization. The solvent, in which the improvement of solubility of the substance by solubilization is sought, may be water.

The term “the body temperature of a mammal” refers to an average temperature prevailing among a group of healthy mammals of the same kind. For example, for humans, the term “the body temperature of a mammal” refers to the temperature of about 37° C.

The term “the liquid-to-gas transition temperature” is defined using the vapor pressure concept. It is well known that for every compound or mixture in a form of a liquid, there always exists some amount of matter in gaseous phase over the liquid. Thus, “the liquid-to-gas transition temperature” is defined as the temperature, at ambient pressure, at which the vapor pressure on the liquid/gas interface is at least about 300 mm Hg.

The term “a stabilizing material” refers to any material which is capable of stabilizing nanodroplets containing lipids, fluorinated compounds, targeting ligands, therapeutic compounds and/or other bioactive agents.

The term “an improved stability” refers to the maintenance and/or preservation of a relatively balanced condition, including maintaining or increasing resistance of the composition against destruction, decomposition, degradation, and the like.

The term “an emulsion” is defined as a colloid system in which both phases are liquids.

The term “a suspension” is defined as a colloid system that has a continuous liquid phase in which a solid is suspended.

The terms “a stable emulsion” and “a stable suspension” are defined as an emulsion or a suspension, respectively, in which the phases do not separate for a substantial period of time.

The term “cross-linked” refers to a chemical structure having chemical links between separate molecular chains to form a three-dimensional network supramolecular system. The material is defined as “substantially cross-linked” if the material's degree of solubility in a solvent is 50% or less of the degree of solubility in the same solvent prior to the process of cross-linking.

### B. Embodiments of the Invention

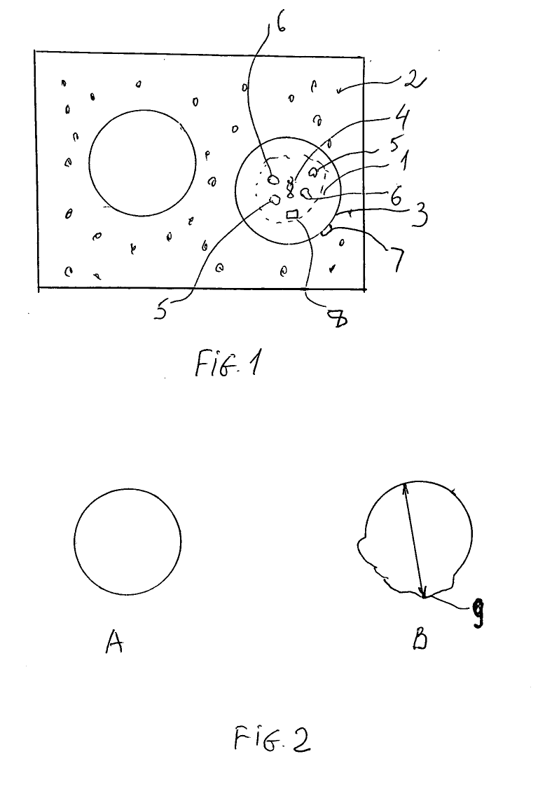

Referring to FIG. 1, there is provided a composition comprising nanodroplets 1 dispersed in an aqueous medium 2 to form a colloid system, such as an emulsion or a suspension. The nanodroplets 1 comprise a lipid 3 defining a void and thus encapsulating a liquid phase 4. The liquid phase 4 comprises a biologically compatible oil 5 and a fluorocarbon composition 6, as defined above. The nanodroplets 1 may optionally comprise a stabilizing material (not shown).

As may be seen from FIG. 1, the biologically compatible oil 5 and the fluorocarbon composition 6 are mixed together. Thus, the biologically compatible oil 5 and the fluorocarbon composition 6 are miscible and may form a true solution. If they do not form a true solution, they may alternatively from a stable colloid system, such as a stable emulsion or stable suspension.

The nanodroplets may further include a therapeutically active compound (drug) 8 that may be solubilized using the biologically compatible oil 5. “Solubilization” of the therapeutically active compound 8 using the biologically compatible oil 5, may include dissolving the therapeutically active compound 8 by the biologically compatible oil 5, or alternatively forming a stable oil/drug colloid system, such as forming a stable suspension of the therapeutically active compound 8 in the biologically compatible oil 5.

The nanodroplets 1 may be generally spherically shaped or may resemble shapes that are not spherically shaped (e.g., may be dimple-shaped). A variety of sizes of the nanodroplets 1 may be present in a sample or dose for administration. For spherically shaped nanodroplets 1, the size of the nanodroplets 1 can be characterized using the mean value of the diameter of the nanodroplets 1 in the sample. Such mean value may be less than about 1 μm, for example, between about 100 nm and about 500 nm. For non-spherical nanodroplets, i.e., imperfectly spherically shaped nanodroplets, the mean value of the quasi-diameter may be used for the size characterization of the nanodroplets.

FIG. 2 demonstrates schematically spherically shaped (A) and non-spherical (B) nanodroplet 1, where FIG. 2 (B) shows the quasi-diameter 9. The mean value of quasi-diameter 9 in a sample may be the same as that of the diameter that is used for characterization of spherically shaped nanodroplets 1, i.e., the quasi-diameter 8 may have the length less than about 1 μm, for example, between about 100 nm and about 500 nm.

Within the nanodroplet 1, the fluorocarbon composition 6 is in the liquid state, at ambient pressure, at a temperature that is equal to, or lower than, the body temperature of a mammal. To be in the liquid state, at ambient pressure, the liquid-to-gas transition temperature of at least one of the fluorinated hydrocarbons forming the fluorocarbon composition 6 is higher that the normal body temperature of a mammal. The normal body temperature of a mammal may be at or below about 50° C., such as at or below about 40° C. (e.g., at or below about 37° C. in case of human patients).

Other fluorinated hydrocarbons, if present in the fluorocarbon composition 6, may be also in the liquid state. Alternatively, some of fluorinated hydrocarbons that are present in the fluorocarbon composition 6 may be in the gaseous state (i.e., may have the liquid-to-gas transition temperature that is lower that the normal body temperature of a mammal, at ambient pressure), so long as the overall fluorocarbon composition 6 is in the liquid state at ambient pressure.

To illustrate, in the fluorocarbon composition 6 that is included within the nanodroplet 1, at least one of the fluorinated hydrocarbons may be a C6 fluorocarbon or higher, as defined above; for example, perfluorohexane may be used. Perfluorohexane and fluorocarbons having hydrocarbon chains with greater than 6 carbon atoms are in liquid state at 37° C. or at a lower temperature at ambient pressure.

Once a C6 fluorocarbon or higher is present in the nanodroplet 1, a C5 fluorocarbon or lower may be further optionally encapsulated in the nanodroplet 1, so long as the overall fluorocarbon composition 6 remains in the liquid state at ambient pressure. One example of a C5 fluorocarbon or lower that may be used is perfluoropentane having the liquid-to-gas transition temperature at ambient pressure of about 30° C.

If a fluorocarbon composition 6 includes both a C6 fluorocarbon or higher (i.e., those that are liquid at ambient conditions) and a C5 fluorocarbon or lower (i.e., those that are gaseous at ambient conditions), those skilled in the art may use common techniques to determine a ratio between the two kinds of fluorocarbons, at which ratio the overall fluorocarbon composition 6 will be in the liquid state at ambient conditions.

The quantity of the fluorocarbon composition 6 present inside the nanodroplet 1 may be between about 0.1 mass % and about 10.0 mass % relative to the quantity of oil 5, for example, about 1.0 mass %, corresponding to fluorinated hydrocarbons:oil ratio (by mass) between about 1:999 and about 1:9, more specifically, between about 1:199 and about 1:19, for example, about 1:99.

A variety of fluorinated hydrocarbons, including perfluorinated hydrocarbons, may be used for making the fluorocarbon composition 6 that is included within the nanodroplets 1 of the present invention. For example, perfluorohexane may be used, including perfluoro-n-hexane, and any isomer thereof. Other perfluorinated hydrocarbons that may be used include perfluoromethane, perfluoroethane, perfluoropropane (any isomer), perfluorobutane (any isomer), perfluorpentane (any isomer), perfluoroheptane (any isomer), perfluorooctane (any isomer), perfluorononane (any isomer), perfluorocyclobutane and mixtures thereof.

Specific examples of some fluorinated compounds, other than perfluorinated hydrocarbons, that may be used for making the fluorocarbon composition 6 include heptafluoropropane (including both 1,1,1,2,3,3,3-heptafluoropropane and 1,1,2,2,3,3,3-heptafluoropropane), perfluorooctyl-bromide, perfluorodecalin, perfluorododecalin, perfluorooctyliodide, perfluorotripropylamine, perfluorotributylamine, and sulfur hexafluoride.

A variety of lipids 3 may be used for making the nanodroplets of the present invention, such as phospholipids, saturated and unsaturated fatty acids, lysolipids, and lipids carrying hydrophilic polymers. For example, dipalmitoylphosphatidylcholine (DPPC) or distearoylphosphatidylcholine (DSPC) may be used. Other examples of lipids that may be used include dioleoylphosphatidylcholine, dimyristoylphosphatidylcholine, dipentademayoylphosphatidylcholine, dilauroylphosphatidylcholine, dioleoylphosphatidylcholine, phosphatidylethanolamines (e.g., dioleoylphosphatidylethanolamine), phosphatidylserine, phosphatidylglycerol, phosphatidylinositol, sphingolipids (e.g., sphingomyelin), glycolipids (e.g., ganglioside GM1 and GM2), glucolipids, sulfatides, glycosphingolipids, phosphatidic acid, lipids bearing polymers (e.g., bearing polyethyleneglycol (“PEGylated lipids”), chitin, hyaluronic acid or polyvinylpyrrolidone), lipids bearing polysaccharides (e.g., bearing sulfonated mono-, di-, oligo- or polysaccharides), cholesterol, cholesterol sulfate, cholesterol hemisuccinate,tocopherol hemisuccinate, lipids with ether and ester-linked fatty acids, polymerized lipids, diacetyl phosphate, stearylamine, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3-β-yloxy)-1-thio-β-D-galactopyranoside, digalactosyldiglyceride, 6-(5-cholesten-3-β-yloxy)hexyl-6-amino-6-deoxy-1-thio-β-D-galacto pyranoside, 6-(5-cholesten-3-β-yloxy)hexyl-6-amino-6-deoxyl-1-thio-α-D-manno pyranoside, 12-(((7′-diethylaminocoumarin-3-yl)carbonyl)methylamino)-octademayoic acid, N-[12-(((7′-diethylaminocoumarin-3-yl)carbonyl)methyl-amino)octademayoyl]-2-aminopalmitic acid, cholesteryl(4′-trimethylammonio)butanoate, N-succinyldioleoylphosphatidylethanolamine, 1,2-dioleoyl-sn-glycerol, 1,2-dipalmitoyl-sn-3-succinylglycerol, 1,3-dipalmitoyl-2-succinylglycerol; 1-hexadecyl-2-palmitoylglycerophosphoethanolamine, palmitoylhomocysteine, and combinations thereof.

Additionally, PEGylated lipids that may be used include poly(ethylene glycol) (PEG)-based lipids having a molecular weight of between about 1,000 Daltons and 10,000 Daltons, for example about 2,000 Daltons, 5,000 Daltons, or 8,000 Daltons. Saturated and unsaturated fatty acids that may be used include molecules that have between 12 carbon atoms and 22 carbon atoms in either linear or branched form. Some examples of specific saturated fatty acids that may be used include, but are not limited to, lauric, myristic, palmitic, and stearic acids. Some examples of specific unsaturated fatty acids that may be used include, but are not limited to, lauroleic, physeteric, myristoleic, palmitoleic, petroselinic, linoleic, and oleic acids. Some examples of specific branched fatty acids that may be used include, but are not limited to, isolauric, isomyristic, isopalmitic, and isostearic acids and isoprenoids

Targeting ligand 7 may be optionally attached to the outer surface of the lipid 3 forming the nanodroplets 1. The concentration of the targeting ligands may range from about 5 mass % to about 40 mass % of the nanoparticle 1, for example, about 5%, about 10%, about 15%, about 200%, about 25%, about 30%, about 35% or about 40% of the nanoparticle 1 by mass. The concentration of the targeting ligands may be about 10 mass % of the total lipid content in the nanodroplet.

One way of attaching the targeting ligand 7 to the lipid 3 may include using physical bonding, e.g., using electrostatic or Van-der-Waals-type bonding, and the like. If desirable, in some cases, chemical bonding may be used for attaching the targeting ligand 7 to the lipid 3, for example via covalent or non-covalent conjugating. Some ways of incorporating the targeting ligand 7 include covalent or non-covalent bonding with one or more of the materials which are included in the compositions, including, for example, lipids, proteins or polymers, as well as any auxiliary stabilizing materials.

For example, targeting ligands 7 in the form of phospholipids, such as acidic phospholipids and/or phospholipids containing phosphorylated serine moieties, may be incorporated into the nanoparticles 1 by agitation or sonication the acidic phospholipids along with the lipid component 3 during the preparation of the nanoparticles. Those having ordinary skill in the art may determine which kinds of ligands are suitable for chemical conjugation given the chemical nature of particular lipid 3 that is used.

As one illustration, targeting ligand 7 having anionic nature may be bound to the lipid 3 taking advantage of the charge that is present on the surface of the lipid layer 3. For instance, a cationic lipid 3 having positively charged groups may be used to complex negatively charged molecules of the targeting ligand 7, thus binding the targeting ligand 7 to the surface of the nanodroplet 1. Alternatively, for example, negatively charged molecules may be used for forming the targeting ligand 7 by binding to the lipid 3 via ester, amide, ether, disulfide or thioester linkages.

Accordingly, biologically active materials, such as peptides, polypeptides, antibodies, or proteins, may be used for creating the targeting ligand 7. Such biologically active materials may be incorporated into the nanodroplets 1 by binding to, or inserting into, or associating with, the lipid layer 3. The successful incorporation via the lipid layer 3 is typically possible if the peptides, polypeptides, antibodies, or proteins to be bound are sufficiently lipophilic. If some peptides, polypeptides, antibodies, or proteins are generally insufficiently lipophilic, sometimes they may be preliminarily derivatized to increase lipophilicity, e.g., with alkyl groups. So derivatized peptides, polypeptides, antibodies, or proteins may then be incorporated into the nanodroplets 1 by binding to the lipid layer 3. Negatively charged peptides may be attached, for example, using cationic lipids or polymers as described above.

If targeting ligand 7 is to be used, the quantity of the ligand to be bonded to the lipid 3 may be between about 0.1 and about 20.0 mass % of the lipid 3. A variety of compounds may be utilized for forming the targeting ligand 7, including bioconjugate analogs, peptides, polypeptides, antibodies, or proteins. For example, the bioconujugate analogs directed to α₆β₁ and α₃β₁ receptors may be used such as the conjugate N,N dialkyl-diaminobutyryl-polyethyleneglycol₃₄₀₀-(2,7-cyclo)-RKRLQVDLSI-NH₂. Other conjugates incorporating moieties derived from phosphatidic acids (e.g., dipalmitoylphosphatidic acid), phosphatidyl serines (e.g., dipalmitoylphosphatidyl serine) or phosphatidylinositols (e.g., dipalmitoylphosphatidylinositol) may be used.

PEG represents one example of a hydrophilic polymer that may be carried by a targeting ligand 7 and the above-described conjugate directed to α₆β₁ and α₃β₁ receptors includes a PEG-derived moiety. In addition to PEG, some other hydrophilic polymer moieties may be used for forming a targeting ligand 7. Some examples of such other hydrophilic polymer moieties include polyalkyleneoxides other than PEG, for example, polypropylene glycol (PPG), polyvinylpyrrolidones, polyvinyl alcohols, polyvinylmethylethers, polyacrylamides, such as, for example, polymethacrylamides, polydimethylacrylamides or polyhydroxypropylmethacrylamides, polyhydroxyethyl acrylates, polyhydroxypropyl methacrylates, polymethyloxazolines, polyethyloxazolines, polyhydroxyethyloxazolines, polyhyhydroxypropyloxazolines, polyphosphazenes, poly (hydroxyalkylcarboxylic acids), polyoxazolidines, polyaspartamide and copolymers thereof.

Thus, in embodiments involving lipid compositions which comprise lipids bearing polymers including, for example, dipalmitoylphosphatidyl ethanolamine-PEG (DPPE-PEG), the targeting ligand may be linked directly to the polymer which is attached to the lipid to provide, for example, a conjugate of DPPE-PEG-TL, where TL is a targeting ligand. PEG may have molecular weight between about 2,000 Daltons and about 10,000 Daltons, for example, 2,000 Daltons, 3,400 Daltons, or 5,000 Daltons. Thus, using the example DPPE-PEG, such as, DPPE-PEG5000 (for PEG having molecular weight of 5,000 Daltons), the conjugate may be represented as DPPE-PEG5000-TL. The hydrophilic polymer used as a linking group may be a bifunctional polymer, for example, bifunctional PEG, such as diamino-PEG. In this case, one end of the PEG group is linked, for example, to a lipid compound, and is bound at the free end to the targeting ligand via an amide linkage. A hydrophilic polymer, for example, PEG, substituted with a terminal carboxylate group on one end and a terminal amino group on the other end, may also be used. These latter bifunctional hydrophilic polymer may be preferred since they possess various similarities to amino acids. Standard peptide methodology may be used to link the targeting ligand to the lipid when utilizing linker groups having two unique terminal functional groups.

For illustrative purposes, if the targeting ligand 7 carries polyethylene glycol (PEG) moiety, one way of binding PEG may be using the phosphate serine moiety through a covalent bond, such as an amide, carbamate, ether or amine linkage. In embodiments involving phosphorylated serine moieties, the resulting targeting ligand may be depicted generically by the formula PEG-P(O)ₓ-serine, where x is 2, 3 or 4. Alternatively, the hydrophilic polymer may be linked to the lipid portion of the targeting ligand. The chemical structure of such embodiments may be depicted as PEG-glycerol-P(O)ₓ-serine, where x is 2, 3 or 4. In these embodiments, the PEG or other polymer may be covalently bonded, for example, through amide, ester, ether, thioester, thioamide or disulfide bonds. In accordance with these embodiments, the distal end of the PEG polymer, i. e., the end of the polymer that is not attached to the serine or glycerol moieties, may be linked or conjugated to other components of the present compositions, for example, other lipids or polymers, stabilizing materials, bioactive agents, and the like. In some embodiments, the distal end of the PEG polymer is attached to a lipid to provide a bioconjugate which may be incorporated into the vesicle walls. Such bioconjugates may be generically depicted by the formula lipid-PEG-P(O)ₓ-serine.

A variety of biocompatible oils 5 may be used for making the nanodroplets 1 of the present invention. Typically, a biocompatible oil 5 that may be used is capable of solubilizing the therapeutically active compound 8. Typically, among the oils useful in the methods and compositions of the present invention, low viscosity oils, may be used, i.e., the oils which may have a viscosity at ambient room temperature ranging from about 1 centipoise to about 4,000 centipoise, and all combinations and subcombinations of ranges and specific viscosities therein, such as between about 1 centipoise and about 2,000 centipoise, for example, between about 1 centipoise and about 1,000 centipoise, more specifically, having viscosities between about 1 centipoise and about 500 centipoise or less. For example, triacetin, diacetin, tocopherol, or mineral oils may be used.

Other examples of biocompatible oils 5 that may be used include such oils are those listed in U.S. Pat. No. 5,633,226, the disclosure of which is hereby incorporated in its entirety by reference herein, and include CAPTEX®200, WHITEPSOL®H-15 and MYVACET®9-45K, hydrogenated cocoa oil, coconut oil, elm seed oil, palm oil, cottonseed oil, soybean oil, parsley seed oil, mustard seed oil, linseed oil, tung oil, pomegranite seed oil, laurel oil, rapeseed oil, corn oil, castor oil, Japanese anise oil, oil of eucalyptus, rose oil, and almond oil.

A variety of therapeutically active compounds 8 may be used for solubilization using the biocompatible oil 5. The concentration of the therapeutically active compounds 8 in the nanoparticle 1 may be between about 0.001 mass % and about 10.0 mass % of the oil content of the nanoparticle. The specific nature of a therapeutically active compound 8 to be used may be determined by the kind of disease or disorder that is intended to be treated. For example, anti-cancer agents may be used as therapeutically active compounds 8, e.g., TAXOL® or paclitaxel, among other kinds of drugs.

Some specific examples of other therapeutically active compounds 8 that may be used include statins, such as lovastatin, pravastatin, simvastatin, cerivastatin, fluvastatin, atrovastatin, eptastatin or mevastatin, antineoplastic agents, such as platinum compounds (e. g., spiroplatin, cisplatin, and carboplatin), methotrexate, adriamycin, mitomycin, ansamitocin, bleomycin, cytosine arabinoside, arabinosyl adenine, mercaptopolylysine, vincristine, busulfan, chlorambucil, melphalan (e. g., PAM, L-PAM or phenylalanine mustard), mercaptopurine, mitotane, procarbazine hydrochloride dactinomycin (actinomycin D), daunorubicin hydrochloride, doxorubicin hydrochloride, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) erwiraa asparaginase, etoposide (VP-16), interferon a-2a, interferon a-2b, teniposide (VM-26), vinblastine sulfate (VLB), vincristine sulfate, bleomycin, bleomycin sulfate, methotrexate, adriamycin, and arabinosyl; blood products such as parenteral iron, hemin, hematoporphyrins and their derivatives; biological response modifiers such as muramyldipeptide, muramyltripeptide, microbial cell wall components, lymphokines (e. g., bacterial endotoxin such as lipopolysaccharide, macrophage activation factor), sub-units of bacteria (such as mycobacteria, corynebacteria), the synthetic dipeptide n-acetyl-muramyl-1-alanyl-d-isoglutamine; anti-fungal agents such as ketoconazole, nystatin, griseofulvin, flucytosine (5-fc), miconazole, amphotericin B, ricin, and p-lactam antibiotics (e. g., sulfazecin); hormones such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide and fludrocortisone acetate; vitamins such as cyanocobalamin neinoic acid, retinoids and derivatives such as retinol palmitate, and a-tocopherol; peptides, such as manganese super oxide dismutase; enzymes such as alkaline phosphatase; anti-allergic agents such as amelexanox; anti-coagulation agents such as phenprocoumon and heparin; circulatory drugs such as propranolol; metabolic potentiators such as glutathione; antituberculars such as para-aminosalicylic acid, isoniazid, capreomycin sulfate cycloserine, ethambutol hydrochloride ethionamide, pyrazinamide, rifampin, and streptomycin sulfate; antivirals such as acyclovir, amantadine azidothymidine (AZT or Zidovudine), ribavirin and vidarabine monohydrate (adenine arabinoside, ara-A); antianginals such as diltiazem, nifedipine, verapamil, erythritol tetranitrate, isosorbide dinitrate, nitroglycerin (glyceryl trinitrate) and pentaerythritol tetranitrate; anticoagulants such as phenprocoumon, heparin; antibiotics such as dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin g, penicillin v, ticarcillin rifampin and tetracycline; antiinflammatories such as diflunisal, ibuprofen, indomethacin, meclofenamate, mefenamic acid, naproxen, oxyphenbutazone, phenylbutazone, piroxicam, sulindac, tolmetin, aspirin and salicylates ; antiprotozoans such as chloroquine, hydroxychloroquine, metronidazole, quinine and meglumine antimonate; antirheumatics such as penicillamine; narcotics such as paregoric; opiates such as codeine, heroin, methadone, morphine and opium; cardiac glycosides such as deslanoside, digitoxin, digoxin, digitalin and digitalis; neuromuscular blockers such as atracurium mesylate, gallamine triethiodide, hexafluorenium bromide, metocurine iodide, pancuronium bromide, succinylcholine chloride (suxamethonium chloride), tubocurarine chloride and vecuronium bromide; sedatives (hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics such as bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics such as droperidol, etomidate, fentanyl citrate with droperidol, ketamine hydrochloride, methohexital sodium and thiopental sodium; and radioactive particles or ions such as strontium, iodide rhenium and yttrium. Other suitable bioactive agents include the camptotheca alkaloids and derivatives thereof including, for example, camptothecin and ester or amide derivatives thereof, particularly at the 7, 9, 10, 11 and 20 ring positions, as well as irinotemay, topotemay and SN-38.

As mentioned above, stabilizing materials may be optionally included within the nanodroplets 1. The stabilizing materials may serve to stabilize the nanodroplets, and to minimize or substantially prevent the escape of gases, gaseous precursors, steroid prodrugs and/or bioactive agents from the nanodroplets. Exemplary stabilizing materials include lipids, proteins, polymers, carbohydrates and surfactants, and may also comprise salts and/or sugars. In certain embodiments, the stabilizing materials may be substantially cross-linked. The stabilizing material may be neutral, positively or negatively charged.

Various techniques and procedures may be used for making the nanodroplet compositions described above. Those having ordinary skill in the art may select the suitable method of fabrication. Without excluding any other method that may be utilized, one method that may be used is described below.

A lipid or a lipid mixture may be combined in a saline solution with a compound to form a targeting ligand, e.g., with a bioconjugate. For example, a 1 mg mL⁻¹ lipid mixture comprising 82 mole % dipalmitoylphosphatidylcholine (DPPC), 8 mole % dipalmitoylphosphatidyl ethanolamine-polyethyleneglycol-3400 (DPPE-PEG₃₄₀₀), and 10 mole % dipalmitoylphosphatidic acid (DPPA), may be added to normal saline followed by addition of 1-5 weight % equivalents of the bioconjugate.

The mixture may then be freeze-thawed approximately four times to yield a colloidal suspension, followed by drying in vacuo. The lipid/bioconjugate mixture may be then reconstituted in an excipient mixture, for example, a mixture comprising normal saline, glycerol, and propylene glycol (8:1:1, v:v:v). Following reconstitution, a fluorinated compound, for example, perfluorohexane may be added to the formulation equivalent to 20% of the volume. The formulation may be then microfluidized for at least 10 passes followed by sizing, using, for example, a Particle Sizing Systems Model 370 sub-micron particle sizer (Particle Sizing Systems, Santa Barbara, Calif.).

### C. EXAMPLES

The following examples are provided to further illustrate the advantages and features of the present invention, but are not intended to limit the scope of the invention.

### Example 1

### Formulating Nanodroplets

40 mL of lipid mixture containing dipalmitoylphosphatidylcholine (DPPC), dipalmitoylphosphatidyl-ethanolamine polyethyleneglycol MW-5000 (DPPE PEG-5000), and dipalmitoylphosphatidic acid (DPPA), 82:10:8, m:m:m, in a 1% w/v solution was added to a beaker and chilled to 0° C. over an ice bath. 10 mL of perfluorohexane was added dropwise into the lipid solution with simultaneous homogenization at 3500 rpm using a Silverson homogenizer. Upon completion of addition of perfluorohexane, the mixture was homogenized an additional 10 minutes. The cold solution was then stored in a refrigerator overnight. The solution was homogenized using an Emulsiflex™ C5 at 25,000 psi for 10 min followed by five successive extrusions through 100 nm polycarbonate filters. The solutions were opaque and white. The droplets were then measured on a Malvern Zetasizer 3000. Dipalmitoylphosphatidylcholine (DPPC), dipalmitoylphosphatidyl-ethanolamine polyethyleneglycol MW-5000 (DPPE PEG5000), and dipalmitoylphosphatidic acid (DPPA), 82:10:8, m:m:m, were dissolved in 10 mL of propylene glycol at 75° C. (total lipid concentration was 0.5 mg/mL or 1 mg/mL). For targeted formulations, the bioconjugate (5-10 wt %) was added and stirred at 75° C. until dissolved. To this solution was added a mixture of 85 mL phosphate buffered saline and 5 mL glycerol. This solution was further formulated into nanodroplets in a microfluidizer.

Paclitaxel was dissolved at a concentration of 60 mg/mL in triacetin with minimal heating at 35° C. and diluted 1:1 with soybean oil. The microfluidizer chamber was cleaned before use by adding de-ionized water up to rim of the chamber. The pump was engaged to cycle the solution through an 87 μm diamond chamber until the chamber was almost empty. Then the pump was turned off and filled again and this process was repeated 4 times. Once the chamber was cleaned, approximately 30 mL of the lipid mixture was added to the chamber. The solution was allowed to cycle through 2 times. Then 10 mls of the lipid mixture was added to the chamber followed by 600 uL of triacetin/soybean oil (1:1) containing paclitaxel and the remaining lipid mixture. The fluidizer was chilled with ice and finally 600 ul of perfluorocarbon mixture (90% perfluorohexane/10% perfluoropentane) was added dropwise. The mixture was then fluidized for 20 minutes with a head pressure of 50 psi. After 20 minutes the solution, now opaque, was removed from the chamber, put into vials, and then sized.

Although the invention has been described with reference to the above examples, it will be understood that modifications and variations are encompassed within the spirit and scope of the invention. Accordingly, the invention is limited only by the following claims.

What is claimed is:

1. A composition, comprising nanodroplets dispersed in an aqueous medium, wherein the nanodroplets include a lipid defining an inner area of the nanodroplets, the inner area comprising:
(a) a biologically compatible oil;
(b) a fluorocarbon composition including one or more fluorinated compounds; and
(c) a therapeutically active compound,
wherein the fluorocarbon composition is in a liquid state at a temperature that is equal to, or lower than, the body temperature of a mammal.

2. The composition according to claim 1, wherein the lipid is selected from the group consisting of dipalmitoylphosphatidylcholine, distearoylphosphatidylcholine, and combinations thereof.

3. The composition according to claim 1, wherein the lipid is selected from the group consisting of dioleoylphosphatidylcholine, dimyristoylphosphatidylcholine, dipentademayoylphosphatidylcholine, dilauroylphosphatidylcholine, dioleoylphosphatidylcholine, phosphatidylethanolamines, phosphatidylserine, phosphatidylglycerol, phosphatidylinositol, sphingolipids, glycolipids, glucolipids, sulfatides, glycosphingolipids, phosphatidic acid, lipids bearing polymers, chitin, hyaluronic acid or polyvinylpyrrolidone), lipids bearing, cholesterol, cholesterol sulfate, cholesterol hemisuccinate,tocopherol hemisuccinate, lipids with ether and ester-linked fatty acids, polymerized lipids, diacetyl phosphate, stearylamine, cardiolipin, phospholipids with short chain fatty acids of 6-8 carbons in length, synthetic phospholipids with asymmetric acyl, 6-(5-cholesten-3-β-yloxy)-1-thio-β-D-galactopyranoside, digalactosyldiglyceride, 6-(5-cholesten-3-β-yloxy)hexyl-6-amino-6-deoxy-1-thio-β-D-galacto pyranoside, 6-(5-cholesten-3-β-yloxy)hexyl-6-amino-6-deoxyl-1-thio-β-D-manno pyranoside, 12-(((7′-diethylaminocoumarin-3-yl)carbonyl)methylamino)-octademayoic acid, N-[12-(((7′-diethylaminocoumarin-3-yl)carbonyl)methyl-amino) octademayoyl]-2-aminopalmitic acid, cholesteryl(4′-trimethylammonio)butanoate, N-succinyldioleoylphosphatidylethanolamine, 1,2-dioleoyl-sn-glycerol, 1,2-dipalmitoyl-sn-3-succinylglycerol, 1,3-dipalmitoyl-2-succinylglycerol, 1-hexadecyl-2-palmitoylglycerophosphoethanolamine, palmitoylhomocysteine, and combinations thereof.

4. The composition according to claim 1, wherein the biologically compatible. oil has a viscosity at ambient room temperature between about 1 centipoise to about 4,000 centipoise.

5. The composition according to claim 4, wherein the biologically compatible oil is selected from the group consisting of triacetin, diacetin, mineral oils, Captex 200, Whitepsol H-15 and MYVACET 9-45K, hydrogenated cocoa oil, coconut oil, elm seed oil, palm oil, cottonseed oil, soybean oil, parsley seed oil, mustard seed oil, linseed oil, tung oil, pomegranite seed oil, laurel oil, rapeseed oil, corn oil, castor oil, Japanese anise oil, oil of eucalyptus, rose oil, and almond oil.

6. The composition according to claim 1, wherein the body temperature is below about 50° C.

7. The composition according to claim 1, wherein the body temperature is below about 40° C.

8. The composition according to claim 1, wherein the body temperature is below about 37° C.

9. The composition according to claim 1, wherein the fluorinated compounds comprise a perfluorinated hydrocarbon.

10. The composition according to claim 9, wherein the perfluorinated hydrocarbon is a perfluorohexane.

11. The composition according to claim 9, wherein the perfluorinated hydrocarbon is selected from a group consisting of perfluoromethane, perfluoroethane, a perfluoropropane, a perfluorobutane, a perfluorpentane, a perfluoroheptane, a perfluorooctane, a perfluorononane, perfluorocyclobutane and combinations thereof.

12. The composition according to claim 1, wherein the fluorinated compound is selected from the group consisting of a heptafluoropropane, perfluorooctyl-bromide, perfluorodecalin, perfluorododecalin, perfluorooctyliodide, perfluorotripropylamine, perfluorotributylamine, sulfur hexafluoride, and combinations thereof.

13. The composition according to claim 1, wherein the therapeutically active compound is an anti-cancer agent.

14. The composition according to claim 13, wherein the anti-cancer agent is selected from a group consisting of taxol and paclitaxel.

15. The composition according to claim 1, wherein the therapeutically active compound is selected from the group consisting of, lovastatin, pravastatin, simvastatin, cerivastatin, fluvastatin, atrovastatin, eptastatin, mevastatin, spiroplatin, cisplatin, carboplatin, methotrexate, adriamycin, mitomycin, ansamitocin, bleomycin, cytosine arabinoside, arabinosyl adenine, mercaptopolylysine, vincristine, busulfan, chlorambucil, melphalan, mercaptopurine, mitotane, procarbazine hydrochloride dactinomycin (actinomycin D), daunorubicin hydrochloride, doxorubicin hydrochloride, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) erwiraa asparaginase, etoposide (VP-16), interferon a-2a, interferon a-2b, teniposide (VM-26), vinblastine sulfate (VLB), vincristine sulfate, bleomycin, bleomycin sulfate, methotrexate, adriamycin, arabinosyl, parenteral iron, hemin, hematoporphyrins, muramyldipeptide, muramyltripeptide, microbial cell wall components, lymphokines, mycobacteria, corynebacteria, the synthetic dipeptide n-acetyl-muramyl-l-alanyl-d-isoglutamine, ketoconazole, nystatin, griseofulvin, flucytosine (5-fc), miconazole, amphotericin B, ricin, p-lactam antibiotics, growth hormone, melanocyte stimulating hormone, estradiol, beclomethasone dipropionate, betamethasone, betamethasone acetate, betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide, fludrocortisone acetate, cyanocobalamin neinoic acid, retinol palmitate, a-tocopherol, as manganese super oxide dismutase, alkaline phosphatase, amelexanox, phenprocoumon, heparin, propranolol, glutathione, para-aminosalicylic acid, isoniazid, capreomycin sulfate cycloserine, ethambutol hydrochloride ethionamide, pyrazinamide, rifampin, streptomycin sulfate, acyclovir, amantadine azidothymidine (AZT or Zidovudine), ribavirin and vidarabine monohydrate (adenine arabinoside, ara-A), diltiazem, nifedipine, verapamil, erythritol tetranitrate, isosorbide dinitrate, nitroglycerin (glyceryl trinitrate), pentaerythritol tetranitrate, phenprocoumon, dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin g, penicillin v, ticarcillin, rifampin, tetracycline, diflunisal, ibuprofen, indomethacin, meclofenamate, mefenamic acid, naproxen, oxyphenbutazone, phenylbutazone, piroxicam, sulindac, tolmetin, aspirin, salicylates, antiprotozoans, chloroquine, hydroxychloroquine, metronidazole, quinine, meglumine antimonite, penicillamine, paregoric, codeine, heroin, methadone, morphine, opium, deslanoside, digitoxin, digoxin, digitalin, digitalis, atracurium mesylate, gallamine triethiodide, hexafluorenium bromide, metocurine iodide, pancuronium bromide, succinylcholine chloride (suxamethonium chloride), tubocurarine chloride, vecuronium bromide, amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam, triazolam, bupivacaine hydrochloride, chloroprocaine hydrochloride, etidocaine hydrochloride, lidocaine hydrochloride, mepivacaine hydrochloride, procaine hydrochloride, tetracaine hydrochloride, droperidol, etomidate, fentanyl citrate with droperidol, ketamine hydrochloride, methohexital sodium and thiopental sodium, radioactive particles or ions such as strontium, iodide rhenium and yttrium, camptothecin and ester or amide derivatives thereof, irinotemay, topotemay and SN-38.

16. The composition according to claim 1, wherein the concentration of the therapeutically active compound in the nanodroplets is between about 0.001 and about 10.0 mass %.

17. The composition according to claim 1, wherein the nanodroplets further comprise a targeting ligand attached to the lipid.

18. The composition according to claim 17, wherein the targeting ligand comprises peptides, polypeptides, antibodies, or proteins.

19. The composition according to claim 17, wherein the targeting ligand includes hydrophilic polymer moieties.

20. The composition according to claim 19, wherein the hydrophilic polymer moieties are selected from a group consisting of polyethylene glycol, polypropylene glycol, polyvinylpyrrolidone, polyvinyl alcohols, polyvinylmethylether, polymethacrylamide, polydimethylacrylamide, polyhydroxypropylmethacrylamide, polyhydroxyethyl acrylate, polyhydroxypropyl methacrylate, polymethyloxazoline, polyethyloxazoline, polyhydroxyethyloxazoline, polyhyhydroxypropyloxazoline, polyphosphazene, poly (hydroxyalkylcarboxylic acids), polyoxazolidine, polyaspartamide and copolymers thereof.

21. The composition according to claim 18, wherein the quantity of the targeting ligand attached to the lipid is between about 0.1 mass % and about 20 mass % of the lipid.

22. The composition according to claim 1, wherein the nanodroplets have a mean diameter between about 100 nm and about 500 nm.

23. The composition according to claim 1, wherein the mass ratio between the fluorinated hydrocarbon(s) and the biologically compatible oil is between about 1:999 and about 1:9.

24. The composition according to claim 23, wherein the mass ratio between the fluorinated hydrocarbon composition and the biologically compatible oil is between about 1:199 and about 1:19.

25. The composition according to claim 24, wherein the mass ratio between the fluorinated hydrocarbon composition and the biologically compatible oil is about 1:99.
